(19) United States
(12) Patent Application Publication
(10) Pub. No.: US 2017/0276692 A1
(43) Pub. Date: Sep. 28, 2017

(54) METHODS OF MONITORING FOR ADHERENCE TO BREXPIPRAZOLE (REXULTI®) THERAPY

(71) Applicant: Ameritox, LLC, Baltimore, MD (US)

(72) Inventors: Gregory L. McIntire, Greensboro, NC (US); Jeffrey Enders, High Point, NC (US); Erin Strickland, Greensboro, NC (US)

(21) Appl. No.: 15/468,885

(22) Filed: Mar. 24, 2017

Related U.S. Application Data
(60) Provisional application No. 62/312,644, filed on Mar. 24, 2016.

(51) Int. Cl. G01N 33/94; G01N 33/493; G01N 33/70

(52) U.S. Cl. CPC G01N 33/94; G01N 33/70; G01N 33/493

(57) ABSTRACT

Methods for helping to monitor subject adherence with a prescribed antipsychotic drug treatment regimen are disclosed.

### PRIORITY CLAIM

This application claims priority to U.S. Provisional Patent Application Ser. No. 62/312,644, filed Mar. 24, 2016, the entire contents of each of which are incorporated herein by reference and relied upon.

### FIELD

The present invention provides methods for helping monitor patient adherence to brexpiprazole therapy in the treatment of Schizophrenia and other Mental Health Disorders.

### SUMMARY

In various embodiments, the present invention provides methods for helping monitor patient adherence to brexpiprazole therapy in the treatment of Schizophrenia and other Mental Health Disorders. In one embodiment, the method involves the use of OPC3952 as a biomarker in urine.

These and other embodiments of the invention will be disclosed in further detail herein below.

### BRIEF DESCRIPTION OF THE FIGURE

FIG. 1 depicts a proposed metabolic pathway for brexpiprazole.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

Brexpiprazole (Rexulti®) is an atypical antipsychotic prescribed for the treatment of schizophrenia. Adherence has been shown, for example by Velligan, et al., (Psychiatric Services, 54:665, 667 (2003)), to be particularly low in patients with schizophrenia. Urine drug testing has been employed by behavioral health clinicians such as described by Baselt, Disposition of Toxic Drugs and Chemicals in Man (2004) to monitor patient adherence through analysis of drugs and their major plasma metabolites. Typical dosing of brexpiprazole is 0.5-4.0 mg/day. It is well absorbed after oral administration with an oral bioavailability of 95%. The mean elimination half-life is 91 hours. Steady state serum concentrations for brexpiprazole are achieved after 10-12 days of dosing.

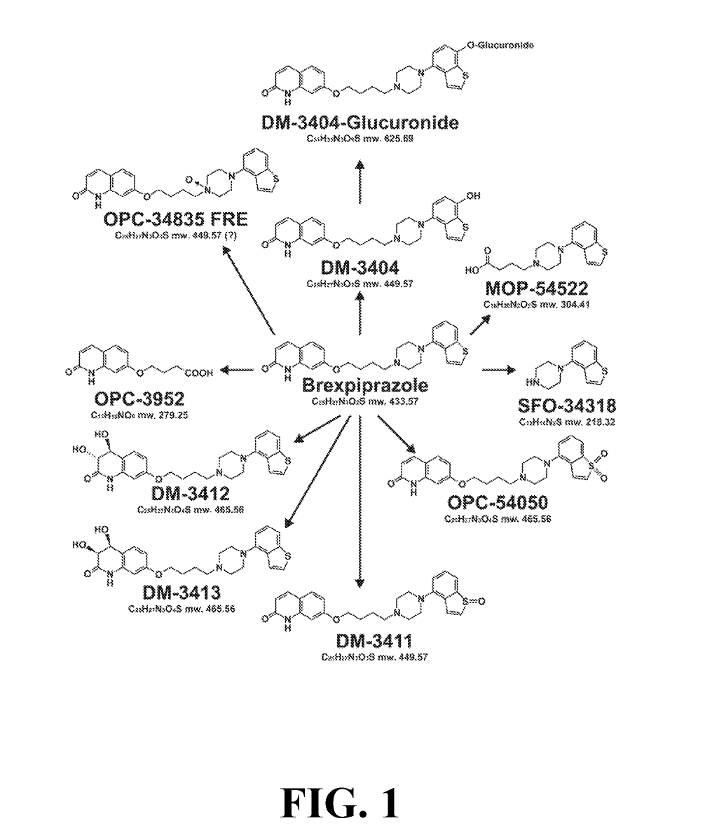

Brexpiprazole is metabolized in the liver primarily by dehydrogenation and hydroxylation by both CYP2D6 and CYP3A4. DM3411 (see FIG. 1) results from oxidation of the sulfur atom in the terminal ring structure to the corresponding sulfoxide. This molecule is not active and represents the major metabolite found in plasma. CYP3A4 mediated N-dealkylation splits brexpiprazole into metabolites, OPC3952 and SF034318. OPC3952 is an acid product with a quinoline moiety and carboxy butyl side chain. Hydroxylation produces metabolites DM3404, DM3412 and DM3413. Secondary biotransformations include N-dealkylation, hydroxylation, and glucuronidation. The proposed metabolic pathway for brexpiprazole is shown in FIG. 1. In plasma, brexpiprazole is the predominant species followed by DM3411 at 23% to 48%, as the major metabolite and OPC3952 as the second metabolite at 0-4%. Brexpiprazole is eliminated by renal and biliary routes in humans. Less than 1% of an oral dose is excreted as unchanged brexpiprazole in urine, with 14% in feces. The N-dealkylation metabolites OPC3952 and SF034318 and related metabolites are excreted predominantly in urine. The larger hydroxylation and dehydrogenation metabolites are excreted predominantly in feces.

In one embodiment, the present disclosure provides a method for monitoring brexpiprazole therapy in a subject. In some embodiments, the method comprises identifying a subject who is prescribed brexpiprazole therapy, measuring a concentration of OPC3952 in urine of the subject, and identifying the subject as non-adherent to brexpiprazole therapy if the concentration of OPC3952 is less than a predetermined level, and as adherent to brexpiprazole therapy if the concentration of OPC3952 is greater than the predetermined level. In some embodiments, the method further comprises counseling the subject on dangers of non-adherence to brexpiprazole therapy if the subject is identified as non-adherent. In some embodiments, the predetermined level is about 25 ng/mL.

In some embodiments, the brexpiprazole therapy includes prescribing to the subject and/or administering to the subject brexpiprazole in an amount selected from about 0.5 mg/day, about 1 mg/day, about 2 mg/day, about 3 mg/day, or about 4 mg/day.

In some embodiments, the method further comprises measuring a concentration of brexpiprazole in urine of the subject, and identifying the subject as non-adherent if the concentrations of brexpiprazole and OPC3952 are both less than predetermined levels of brexpiprazole and OPC3952, respectively. In some embodiments the predetermined level of brexpiprazole is 5 ng/mL. In some embodiments, the predetermined level of OPC3952 is 25 ng/mL.

In some embodiments, the method further comprises measuring a concentration of DM3411 in urine of the subject, and identifying the subject as non-adherent if the concentrations of DM3411 and OPC3952 are both less than predetermined levels of DM3411 and OPC3952, respectively. In some embodiments, the predetermined level of DM3411 is about 5 ng/mL. In some embodiments, the predetermined level of OPC3952 is about 25 ng/mL.

In some embodiments, the method comprises measuring concentrations of brexpiprazole, DM3411 and OPC3952 in urine of the subject, and identifying the subject as non-adherent if the concentrations of brexpiprazole, DM3411 and OPC3952 are each less than predetermined levels of brexpiprazole, DM3411 and OPC3952, respectively. In some embodiments, the predetermined level of brexpiprazole is about 5 ng/mL. In some embodiments, the predetermined level of DM3411 is about 5 ng/mL. In some embodiments, the predetermined level of OPC3952 is about 25 ng/mL.

In some embodiments, the method comprises measuring concentrations of brexpiprazole, DM3411 and OPC3952 in urine of the subject, and identifying the subject as non-adherent if the concentrations of any one, any two, or all three of brexpiprazole, DM3411 and OPC3952 are less than predetermined levels of brexpiprazole, DM3411 and OPC3952, respectively. In some embodiments, the predetermined level of brexpiprazole is about 0.05 ng/mL. In some embodiments, the predetermined level of DM3411 is about 0.05 ng/mL. In some embodiments, the predetermined level of OPC3952 is about 0.25 ng/mL.

The concentration of any analyte described in the present disclosure may be determined (e.g., measured) by any suitable quantification method. In some embodiments, the concentration of an analyte (e.g., brexpiprazole DM3411, and/or OPC3952) is measured by tandem liquid chromatography-mass spectrometry-mass spectrometry (“LC/MSMS”). In some embodiments, the concentration of an analyte (e.g., brexpiprazole, DM3411, and/or OPC3952) is measured by tandem liquid chromatography-High Resolution mass spectrometry (Time of Flight (TOF)) (“LC/MS(TOF)”). In some embodiments, the concentration of an analyte (e.g., brexpiprazole, DM3411, and/or OPC3952) is measured by direct injection mass spectrometry-mass spectrometry (“LC/MSMS”) e.g., such systems as RapidFire™ (Agilent) or Laser Diode Thermal Desorption (“LDTD”) (Phytronix) to introduce samples into the mass spectrometer; either triple quadrupole (i.e., MSMS) or exact mass (i.e., Time of Flight, Orbitrap, etc.) instruments.

In some embodiments, the method further comprises measuring a creatinine level in urine of the subject and normalizing the OPC3952 concentration as a function of the creatinine concentration, wherein the predetermined level comprises a ratio of the OPC3952 concentration to the creatinine concentration.

In some embodiments, the method further comprises measuring a specific gravity value of the urine of the subject, and normalizing the OPC3952 concentration as a function of the specific gravity value, wherein the predetermined level comprises a ratio of the OPC3952 concentration to the specific gravity value.

In some embodiments, the method further comprises measuring a creatinine level in urine of the subject and normalizing the brexpiprazole concentration as a function of the creatinine concentration, wherein the predetermined level comprises a ratio of the brexpiprazole concentration to the creatinine concentration.

In some embodiments, the method further comprises measuring a specific gravity value of the urine of the subject, and normalizing the brexpiprazole concentration as a function of the specific gravity value, wherein the predetermined level comprises a ratio of the brexpiprazole concentration to the specific gravity value.

In some embodiments, the method further comprises measuring a creatinine level in urine of the subject and normalizing the DM3411 concentration as a function of the creatinine concentration, wherein the predetermined level of the DM3411 comprises a ratio of the DM3411 concentration to the creatinine concentration.

In some embodiments, the method further comprises measuring a specific gravity value of the urine of the subject, and normalizing the DM3411 concentration as a function of the specific gravity value, wherein the predetermined level of the DM3411 comprises a ratio of the DM3411 concentration to the specific gravity value.

In some embodiments, the method further comprises measuring a creatinine value in urine of the subject and a specific gravity value of urine of the subject and normalizing the brexpiprazole, DM3411 and/or OPC3952 (“analyte”) concentration(s) as a function of the creatinine and specific gravity values, wherein the predetermined level of the analyte comprises a ratio of the analyte concentration to the creatinine and specific gravity values.

In some embodiments, the method further comprises generating a normal distribution of OPC3952 in brexpiprazole subjects using the log(OPC3952 concentration) from a plurality of measured OPC3952 concentrations in urine from a plurality of subjects, for example a plurality of subjects known to be adherent to brexpiprazole therapy. In some embodiments, the method further comprises identifying a concentration of OPC3952 in urine of a second (test) subject as normal if it falls within the normal distribution (e.g., within 2 standard deviations of the mean) of OPC3952 of the plurality of known adherent brexpiprazole subjects, and/or identifying the concentration of OPC3952 in urine of the second (test) subject as not normal if it falls outside the normal distribution of OPC3952 in the plurality of known adherent brexpiprazole subjects. In some embodiments, the concentration of OPC3952 in urine of the second (test) subject is incorporated into the distribution model, that is, the OPC3952 concentration from the second (test) subject is used along with the OPC3952 concentrations from the plurality of brexpiprazole subjects to generate a second, refined normal distribution of OPC3952 in brexpiprazole subjects.

In some embodiments, the method further comprises generating a normal distribution of brexpiprazole in brexpiprazole subjects using the log(brexpiprazole concentration) from a plurality of measured brexpiprazole concentrations in urine from a plurality of subjects, for example a plurality of subjects known to be adherent to brexpiprazole therapy. In some embodiments, the method further comprises identifying a concentration of brexpiprazole in urine of a second (test) subject as normal if it falls within the normal distribution (e.g., within 2 standard deviations of the mean) of brexpiprazole of the plurality of known adherent brexpiprazole subjects, and/or identifying the concentration of brexpiprazole in urine of the second (test) subject as not normal if it falls outside the normal distribution of brexpiprazole in the plurality of known adherent brexpiprazole subjects. In some embodiments, the concentration of brexpiprazole in urine of the second (test) subject is incorporated into the distribution model, that is, the brexpiprazole concentration from the second (test) subject is used along with the brexpiprazole concentrations from the plurality of brexpiprazole subjects to generate a second, refined normal distribution of brexpiprazole in brexpiprazole subjects.

In some embodiments, the method further comprises generating a normal distribution of DM3411 in brexpiprazole subjects using the log(DM3411 concentration) from a plurality of measured DM3411 concentrations in urine from a plurality of subjects, for example a plurality of subjects known to be adherent to brexpiprazole therapy. In some embodiments, the method further comprises identifying a concentration of DM3411 in urine of a second (test) subject as normal if it falls within the normal distribution (e.g., within 2 standard deviations of the mean) of DM3411 of the plurality of known adherent brexpiprazole subjects, and/or identifying the concentration of DM3411 in urine of the second (test) subject as not normal if it falls outside the normal distribution of DM3411 in the plurality of known adherent brexpiprazole subjects. In some embodiments, the concentration of DM3411 in urine of the second (test) subject is incorporated into the distribution model, that is, the DM3411 concentration from the second (test) subject is used along with the DM3411 concentrations from the plurality of brexpiprazole subjects to generate a second, refined normal distribution of DM3411 in brexpiprazole subjects.

In some embodiments, the method comprises measuring a concentration of OPC3952 and a concentration of brexpiprazole in urine of the subject, and identifying the subject as a poor brexpiprazole metabolizer if a ratio of the OPC3952 concentration to the brexpiprazole concentration is no greater than about 100, no greater than about 25, or no greater than about 5.

Any method disclosed herein may additionally comprise counseling a subject on dangers of non-adherence to brexpiprazole therapy if the subject is identified as non-adherent.

In some embodiments, the method further comprises modifying the subject's prescribed dose of brexpiprazole if the subject is identified as being non-adherent with the prescribed brexpiprazole therapy. In some embodiments, the method further comprises discontinuing brexpiprazole therapy in the subject if the subject is identified as being non-adherent. In some embodiments, the method further comprises replacing brexpiprazole therapy in the subject with a new therapeutic regimen if the subject is identified as being non-adherent. In some embodiments, the method further comprises discontinuing brexpiprazole therapy in the subject and increasing a dose of a second anti-psychotic drug in the subject if the subject is identified as being non-adherent. In some embodiments, the method further comprises maintaining (e.g., not modifying or not significantly modifying) the subject's prescribed dose of brexpiprazole if the subject is identified as being adherent with the prescribed brexpiprazole therapy.

In some embodiments, the method further comprises determining a clinical effect of a subject's brexpiprazole therapy, the method comprising identifying a subject who is prescribed brexpiprazole therapy, measuring a concentration of one or more of OPC3952, brexpiprazole, and DM3411 in urine of the subject, identifying the subject as adherent to brexpiprazole therapy if the concentration of OPC3952, brexpiprazole and/or DM3411 falls within a normal distribution of OPC3952, brexpiprazole and/or DM3411 (respectively), identifying an inadequate clinical response to the prescribed brexpiprazole therapy in the subject, and replacing the prescribed brexpiprazole therapy with a different anti-psychotic drug therapy.

### EXAMPLES

### Example 1

A method for monitoring brexpiprazole therapy in a subject, the method comprising:

identifying a subject who is prescribed brexpiprazole therapy;

measuring a concentration of OPC3952 in urine of the subject; and

identifying the subject as non-adherent to brexpiprazole therapy if the concentration of OPC3952 is less than a predetermined level, and as adherent to brexpiprazole therapy if the concentration of OPC3952 is greater than the predetermined level.

### Example 2

The method of Example 1 further comprising counseling the subject on dangers of non-adherence to brexpiprazole therapy if the subject is identified as non-adherent.

### Example 3

The method of Example 1, wherein the predetermined level is 25 ng/mL.

### Example 4

The method of Example 1, wherein the brexpiprazole therapy comprises prescribing to the subject brexpiprazole in an amount selected from the group consisting of: 2 mg/day, 5 mg/day, 10 mg/day, 15 mg/day, 20 mg/day, or 30 mg/day.

### Example 5

The method of Example 1, wherein the concentration of OPC3952 is measured by LC/MSMS.

### Example 6

The method of Example 1, wherein the concentration of OPC3952 is measured by LC/MS(TOF).

### Example 7

The method of Example 1, wherein the concentration of OPC3952 is measured by direct administration to the mass spectrometer.

### Example 8

The method of Example 7, wherein the direct administration comprises Rapidfire or LDTD.

### Example 9

The method of Example 1 further comprising:
measuring a concentration of brexpiprazole in urine of the subject,

wherein the subject is identified as non-adherent if the concentrations of brexpiprazole and OPC3952 are both less than predetermined levels of brexpiprazole and OPC3952, respectively.

### Example 10

The method of Example 9, wherein the predetermined level of brexpiprazole is 5 ng/mL.

### Example 11

The method of Example 9, wherein the predetermined level of OPC3952 is 25 ng/mL.

### Example 12

The method of Example 1 further comprising:
measuring a concentration of DM3411 in urine of the subject,

wherein the subject is identified as non-adherent if the concentrations of DM3411 and OPC3952 are both less than predetermined levels of DM3411 and OPC3952, respectively.

### Example 13

The method of Example 12, wherein the predetermined level of DM3411 is 5 ng/mL.

### Example 14

The method of Example 12, wherein the predetermined level of OPC3952 is 25 ng/mL.

### Example 15

The method of Example 12, wherein the concentration of OPC3952 is measured by LC/MSMS.

### Example 16

The method of Example 9 further comprising:
measuring a concentration of DM3411 in urine of the subject,

wherein the subject is identified as non-adherent if the concentrations of brexpiprazole, DM3411 and OPC3952 are each less than predetermined levels of brexpiprazole, DM3411 and OPC3952, respectively.

### Example 17

The method of Example 16, wherein the predetermined level of DM3411 is 5 ng/mL.

### Example 18

The method of Example 16, wherein the predetermined level of brexpiprazole is 5 ng/mL.

### Example 19

The method of Example 16, wherein the predetermined level of OPC3952 is 25 ng/mL.

### Example 20

The method of Example 16, wherein the concentration of OPC3952 is measured by LC/MSMS.

### Example 21

The method of Example 1 further comprising:
measuring a creatinine level in urine of the subject and/or a specific gravity value of the urine of the subject; and
normalizing the OPC3952 concentration as a function of the creatinine concentration and/or the specific gravity value,

wherein the predetermined level comprises a ratio of the OPC3952 concentration to the creatinine concentration and/or the specific gravity value.

### Example 22

The method of Example 21 further comprising:
measuring an brexpiprazole concentration in urine of the subject; and normalizing the brexpiprazole concentration as a function of the creatinine concentration and/or the specific gravity value,

wherein the predetermined level comprises a ratio of the OPC3952 concentration and/or the brexpiprazole concentration to the creatinine concentration and/or the specific gravity value.

### Example 23

The method of Example 21 further comprising:
measuring a DM3411 concentration in urine of the subject; and
normalizing the DM3411 concentration as a function of the creatinine concentration and/or the specific gravity value,

wherein the predetermined level of the DM3411 comprises a ratio of the DM3411 concentration to the creatinine concentration and/or the specific gravity value.

### Example 24

The method of Example 1 further comprising:
generating a normal distribution of OPC3952 in brexpiprazole subjects using a log of a plurality of OPC3952 concentrations in urine of a plurality of subjects.

### Example 25

The method of Example 24 further comprising:
identifying a concentration of OPC3952 in urine of the subject as normal if it falls within the normal distribution of OPC3952 in brexpiprazole subjects; and/or
identifying the concentration of OPC3952 in urine of the subject as not normal if it falls outside the normal distribution of OPC3952 in brexpiprazole subjects.

### Example 26

The method of Example 25, further comprising generating a second, refined normal distribution of OPC3952 in brexpiprazole subjects using the OPC3952 concentration in urine of the subject if it is identified as normal.

### Example 27

The method of Example 1 further comprising:
measuring an brexpiprazole concentration in urine of each of a plurality of brexpiprazole subjects; and
generating a normal distribution of brexpiprazole in brexpiprazole subjects using a log of a plurality of brexpiprazole concentrations in urine of the plurality of subjects.

### Example 28

The method of Example 27 further comprising:
measuring an brexpiprazole concentration in the subject; and
identifying the brexpiprazole concentration in urine of the subject as normal if it falls within the normal distribution of brexpiprazole in brexpiprazole subjects; and/or
identifying the brexpiprazole concentration in urine of the subject as not normal if it falls outside the normal distribution of brexpiprazole in brexpiprazole subjects.

### Example 29

The method of Example 27 further comprising generating a second, refined normal distribution of brexpiprazole in brexpiprazole subjects using the brexpiprazole concentration in urine of the subject if it is identified as normal.

### Example 30

The method of Example 1 further comprising:
measuring a DM3411 concentration in urine of each of a plurality of aripiprazole subjects; and
generating a normal distribution of DM3411 in brexpiprazole subjects using a log of the DM3411 concentrations in urine of the plurality of brexpiprazole subjects.

### Example 31

The method of Example 30 further comprising:
measuring a DM3411 concentration in the subject; and

identifying the DM3411 concentration as normal if it falls within the normal distribution of DM3411 in brexpiprazole subjects; and/or
identifying the DM3411 concentration as not normal if it falls outside the normal distribution of DM3411 in brexpiprazole subjects.

### Example 32

The method of Example 31 further comprising generating a second, refined normal distribution of DM3411 in brexpiprazole subjects using the DM3411.

### Example 33

The method of Example 1 further comprising:
measuring a concentration of brexpiprazole in urine of the subject; and
identifying a subject as a poor brexpiprazole metabolizer if a ratio of the OPC3373 concentration to the brexpiprazole concentration is no greater than about 100.

### Example 34

The method of Example 33, wherein the ratio of the OPC3952 concentration to the brexpiprazole concentration is no greater than about 25.

### Example 35

The method of Example 33, wherein the ratio of the OPC3952 concentration to the brexpiprazole concentration is no greater than about 5.

### Example 36

The method of any preceding Example further comprising discontinuing the brexpiprazole therapy in the subject if the subject is identified as being non-adherent.

### Example 37

The method of any preceding Example further comprising replacing the brexpiprazole therapy in the subject with a new therapeutic regimen if the subject is identified as being non-adherent.

### Example 38

The method of any preceding Example further comprising discontinuing the brexpiprazole therapy in the subject and increasing a dose of a second anti-psychotic drug in the subject if the subject is identified as being non-adherent.

### Example 39

The method of any preceding Example further comprising maintaining (e.g., not modifying or not significantly modifying) the subject's prescribed dose of brexpiprazole if the subject is identified as being adherent with the prescribed brexpiprazole therapy.

What is claimed is:

1. A method for monitoring brexpiprazole therapy in a subject, the method comprising:
identifying a subject who is prescribed brexpiprazole therapy;
measuring a concentration of OPC3952 in urine of the subject; and
identifying the subject as non-adherent to brexpiprazole therapy if the concentration of OPC3952 is less than a predetermined level, and as adherent to brexpiprazole therapy if the concentration of OPC3952 is greater than the predetermined level.

2. The method of claim 1 further comprising counseling the subject on dangers of non-adherence to brexpiprazole therapy if the subject is identified as non-adherent.

3. The method of claim 1, wherein the predetermined level is 25 ng/mL.

4. The method of claim 1, wherein the brexpiprazole therapy comprises prescribing to the subject brexpiprazole in an amount selected from the group consisting of: 2 mg/day, 5 mg/day, 10 mg/day, 15 mg/day, 20 mg/day, or 30 mg/day.

5. The method of claim 1, wherein the concentration of OPC3952 is measured by LC/MSMS or LC/MS(TOF).

6. The method of claim 1 further comprising:
measuring a concentration of brexpiprazole in urine of the subject, wherein the subject is identified as non-adherent if the concentrations of brexpiprazole and OPC3952 are both less than predetermined levels of brexpiprazole and OPC3952, respectively.

7. The method of claim 6, wherein the predetermined level of brexpiprazole is 5 ng/mL.

8. The method of claim 1 further comprising:
measuring a concentration of DM3411 in urine of the subject,
wherein the subject is identified as non-adherent if the concentrations of DM3411 and OPC3952 are both less than predetermined levels of DM3411 and OPC3952, respectively.

9. The method of claim 8, wherein the predetermined level of DM3411 is 5 ng/mL.

10. The method of claim 6 further comprising:
measuring a concentration of DM3411 in urine of the subject, wherein the subject is identified as non-adherent if the concentrations of brexpiprazole, DM3411 and OPC3952 are each less than predetermined levels of brexpiprazole, DM3411 and OPC3952, respectively.

11. The method of claim 10, wherein the predetermined level of DM3411 is 5 ng/mL.

12. The method of claim 1 further comprising:
measuring a creatinine level in urine of the subject and/or a specific gravity value of the urine of the subject; and
normalizing the OPC3952 concentration as a function of the creatinine concentration and/or the specific gravity value,
wherein the predetermined level comprises a ratio of the OPC3952 concentration to the creatinine concentration and/or the specific gravity value.

13. The method of claim 12 further comprising:
measuring an brexpiprazole concentration in urine of the subject; and normalizing the brexpiprazole concentration as a function of the creatinine concentration and/or the specific gravity value,
wherein the predetermined level comprises a ratio of the OPC3952 concentration and/or the brexpiprazole concentration to the creatinine concentration and/or the specific gravity value.

14. The method of claim 12 further comprising:
measuring a DM3411 concentration in urine of the subject; and
normalizing the DM3411 concentration as a function of the creatinine concentration and/or the specific gravity value,
wherein the predetermined level of the DM3411 comprises a ratio of the DM3411 concentration to the creatinine concentration and/or the specific gravity value.

15. The method of claim 1 further comprising:
generating a normal distribution of OPC3952 in brexpiprazole subjects using a log of a plurality of OPC3952 concentrations in urine of a plurality of subjects.

16. The method of claim 15 further comprising:
identifying a concentration of OPC3952 in urine of the subject as normal if it falls within the normal distribution of OPC3952 in brexpiprazole subjects; and/or
identifying the concentration of OPC3952 in urine of the subject as not normal if it falls outside the normal distribution of OPC3952 in brexpiprazole subjects.

17. The method of claim 16, further comprising generating a second, refined normal distribution of OPC3952 in brexpiprazole subjects using the OPC3952 concentration in urine of the subject if it is identified as normal.

18. The method of claim 1 further comprising:
measuring an brexpiprazole concentration in urine of each of a plurality of brexpiprazole subjects;
generating a normal distribution of brexpiprazole in brexpiprazole subjects using a log of a plurality of brexpiprazole concentrations in urine of the plurality of subjects;
measuring an brexpiprazole concentration in the subject; and
identifying the brexpiprazole concentration in urine of the subject as normal if it falls within the normal distribution of brexpiprazole in brexpiprazole subjects; and/or
identifying the brexpiprazole concentration in urine of the subject as not normal if it falls outside the normal distribution of brexpiprazole in brexpiprazole subjects.

19. The method of claim 1 further comprising:
measuring a DM3411 concentration in urine of each of a plurality of aripiprazole subjects;
generating a normal distribution of DM3411 in brexpiprazole subjects using a log of the DM3411 concentrations in urine of the plurality of brexpiprazole subjects;
measuring a DM3411 concentration in the subject; and
identifying the DM3411 concentration as normal if it falls within the normal distribution of DM3411 in brexpiprazole subjects; and/or
identifying the DM3411 concentration as not normal if it falls outside the normal distribution of DM3411 in brexpiprazole subjects.

20. The method of claim 1 further comprising discontinuing the brexpiprazole therapy in the subject if the subject is identified as being non-adherent.
